(19) United States
(12) Patent Application Publication
(10) Pub. No.: US 2025/0000336 A1
(43) Pub. Date: Jan. 2, 2025

(54) MODULAR BODY GARMENT FOR ENDOSCOPIC PROCEDURES

(71) Applicant: ColoWrap, LLC., Durham, NC (US)

(72) Inventors: James HATHORN, Durham, NC (US); Anjana Mohanty, Durham, NC (US); Wayne Henderson, Chapel Hill, NC (US); Catherine Froelicher, Raleigh, NC (US)

(21) Appl. No.: 18/759,873

(22) Filed: Jun. 29, 2024

Related U.S. Application Data
(60) Provisional application No. 63/511,620, filed on Jun. 30, 2023.

(51) Int. Cl. A61B 1/00; A61B 1/31

(52) U.S. Cl. CPC A61B 1/00135; A61B 1/00121; A61B 1/31

(57) ABSTRACT

A method and apparatus for applying pressure to a patient's abdomen during an endoscopy procedure. The apparatus comprising a sleeve configured to surround at least a portion of the abdomen of the patient. The sleeve comprises a front portion comprising a first material, a rear portion comprising a second material having a different elasticity than the front portion, and one or more anchor regions on at least the front portion or the rear portion. The sleeve comprises a first anchor region of the one or more anchor regions along a left region of the sleeve and is configured to removably couple with a first end of one or more compression apparatuses, wherein at least a second anchor region of the one or more anchor regions along the rear portion is configured to removably couple with a second end of each of the one or more compression apparatuses.

### CROSS REFERENCE TO RELATED APPLICATION(S)

This application claims the benefit of and priority to U.S. Provisional Application Ser. No. 63/511,620, entitled “Modular Body Garment for Endoscopic Procedures” and filed on Jun. 30, 2023, which is expressly incorporated by reference herein in its entirety.

### INTRODUCTION

A colonoscopy is an example of an endoscopy procedure including an examination of the large intestine or colon through the use of a colonoscope. A colonoscope is a flexible, tube-like inspection device having a camera at its end. Colonoscopies are performed for a variety of medical reasons including detection of inflamed tissue, ulcers, abnormal growths or polyps, and colorectal cancer. Colonoscopy is increasingly used as a screening tool to detect colorectal cancer.

During a colonoscopy, as an example of an endoscopy procedure, a colonoscope is inserted into a patient's rectum and then advanced to the beginning of the colon (an area known as the cecum) in order to examine the lining of the large intestine. The efficiency and accuracy of this procedure is largely dependent on the ease with which the colonoscope can be advanced. During the procedure, the colon may become over-distended or flopped in unnatural directions creating loops that hinder the advancement of the colonoscope and resulting in patient discomfort, longer examination times, and potentially inaccurate or incomplete screenings.

Currently, the difficulty in advancing the scope is addressed by the application of manual pressure by a technician to manually support the patient's colon. The application of manual pressure is time-consuming and varies depending on the particular technician's strength, technique, endurance, and training. In order to apply differential pressure or to change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure places a physical toll on the technician.

### SUMMARY

In an aspect of the disclosure, a method and apparatus for applying pressure to the abdomen of a patient is provided to ease the passage of an endoscope during procedures used to examine the bowels including colonoscopy, sigmoidoscopy, and enteroscopy. Aspects presented herein exert both broad, uniform lower abdominal pressure as well as additional, location-specific pressure upon specific parts of the colon including the sigmoid and transverse, and/or small bowel to assist with the advancement, withdrawal, and/or visualization as part of an endoscopy, colonoscopy, sigmoidoscopy, or enteroscopy procedure. Aspects presented herein may assist in preventing and reducing intestinal looping, eliminating the need for the application of manual pressure, improving patient safety, comfort, and satisfaction, and preventing musculoskeletal injury to endoscopy healthcare providers. Aspects may improve visualization and/or withdrawal portions of the procedure.

Aspects include a sleeve, or endoscopy garment, configured to surround at least a portion of an abdomen of a patient and at least one anchor region. The sleeve may comprise a front portion, a rear portion, and one or more anchor regions. The front portion may be comprised of a first material and the rear portion may be comprised of a second material that has a different elasticity than the first material. The one or more anchor regions may be on the front portion or the rear portion. An attachment extension may be adjacent or interposed between the front portion or the rear portion and configured to removably couple with a first end of one or more external compression apparatuses, while the one or more anchor regions are configured to be removably coupled with a second end of the one or more external compression apparatuses when the compression apparatuses are placed over the abdomen of the patient between the at least one attachment extension and the one or more anchor regions.

Additional advantages and novel features of aspects of the present invention will be set forth in part in the description that follows, and in part will become more apparent to those skilled in the art upon examination of the following or upon learning by practice thereof.

### BRIEF DESCRIPTION OF THE DRAWINGS

FIG. 1A is a schematic view of a colon with an endoscope (also known as a colonoscope for colonoscopy procedures) partially inserted therein.

FIG. 1B is a schematic view of a colon in which a sigmoid loop has developed due to an attempt to advance the endoscope against an unsupported colon wall;

FIG. 1C is a schematic view of a colon showing the application of manual pressure to the colon to facilitate insertion of an endoscope.

FIG. 2A is a front view of the modular body garment, in accordance with aspects presented herein.

FIG. 2B is a rear view of the modular body garment, in accordance with aspects presented herein.

FIG. 3A is a front view of a compression apparatus, in accordance with aspects presented herein.

FIG. 3B is a rear view of the compression apparatus, in accordance with aspects presented herein.

FIG. 4A is a front view of the modular body garment, in accordance with aspects presented herein.

FIG. 4B is a rear view of the modular body garment, in accordance with aspects presented herein.

FIG. 5A is a front view of a modular body garment, in accordance with aspects presented herein.

FIG. 5B is a rear view of the modular body garment, in accordance with aspects presented herein.

FIG. 6A is a front view of a modular body garment, in accordance with aspects presented herein.

FIG. 6B is a rear view of the modular body garment, in accordance with aspects presented herein.

FIG. 7 is a partial overhead view of a modular body garment, in accordance with aspects presented herein.

FIG. 8A is a front view of a modular body garment, in accordance with aspects presented herein.

FIG. 8B is a rear view of the modular body garment, in accordance with aspects presented herein.

FIG. 9A is a front view of a modular body garment, in accordance with aspects presented herein.

FIG. 9B is another front view of a modular body garment, in accordance with aspects presented herein.

FIG. 9C is a rear view of the modular body garment, in accordance with aspects presented herein.

FIG. 10A is a front view of a modular body garment, in accordance with aspects presented herein.

FIG. 10B is a rear view of a modular body garment, in accordance with aspects presented herein.

FIG. 11A is a front view of a modular body garment, in accordance with aspects presented herein.

FIG. 11B is a rear view of a modular body garment, in accordance with aspects presented herein.

FIG. 12A is a front view of a modular body garment, in accordance with aspects presented herein.

FIG. 12B is a rear view of a modular body garment, in accordance with aspects presented herein.

FIG. 13 illustrates examples of a compression board positioned under a patient to allow for the application of targeted compression during an endoscopy procedure, in accordance with aspects presented herein.

FIG. 14 illustrate example aspects of a compression board and compression apparatus having a hooking mechanism, in accordance with aspects presented herein.

FIG. 15A and FIG. 15B illustrate examples aspects in which a compression board may be used to provide added leverage for a compression apparatus applied in connection with an endoscopy garment, in accordance with aspects presented herein.

FIG. 16A illustrates an example of a compression board, in accordance with aspects presented herein.

FIG. 17A illustrates examples aspects of a compression apparatus that includes a hook or attachment piece, in accordance with aspects presented herein.

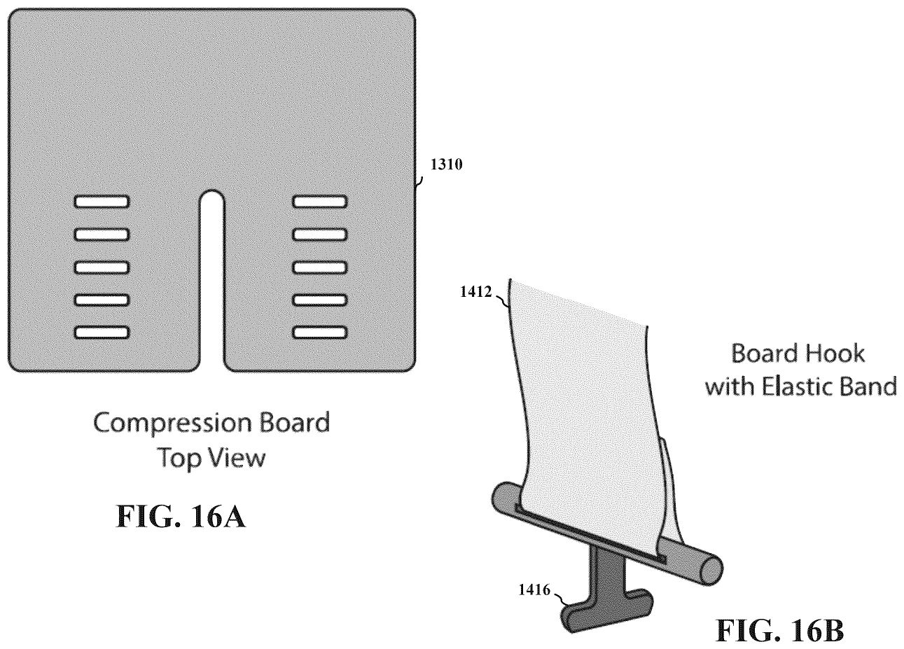

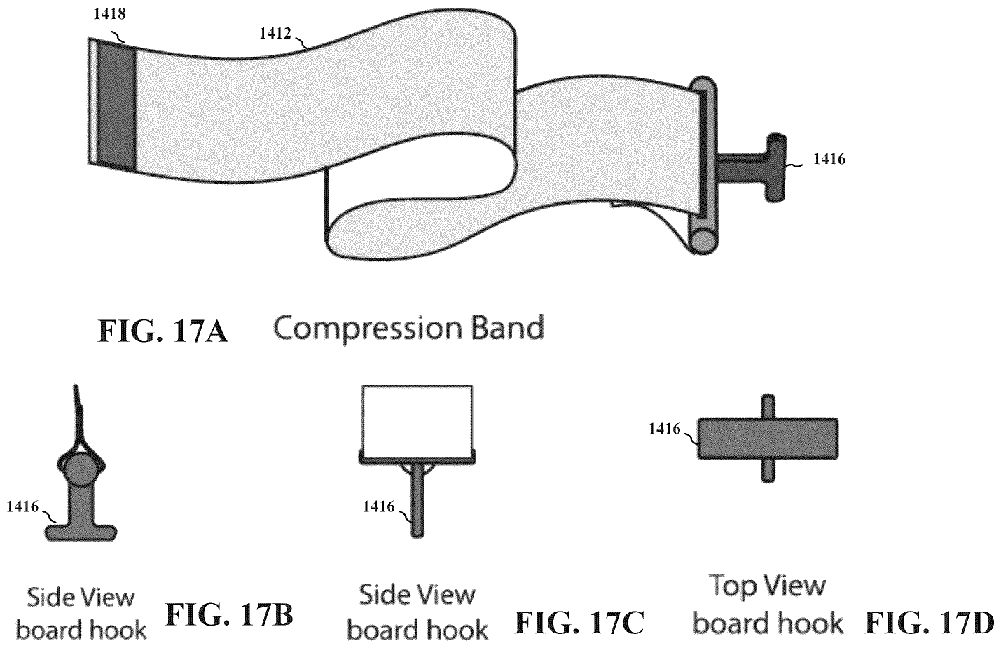

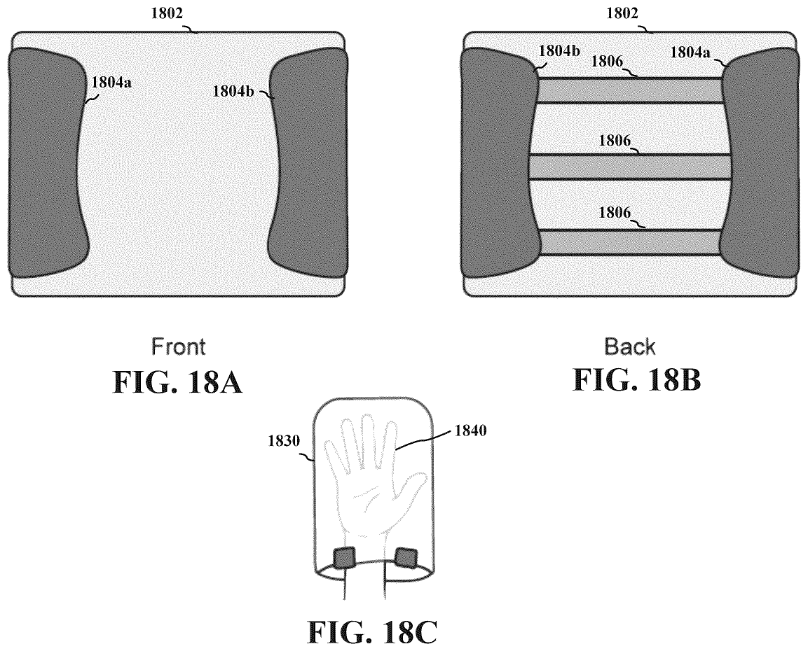

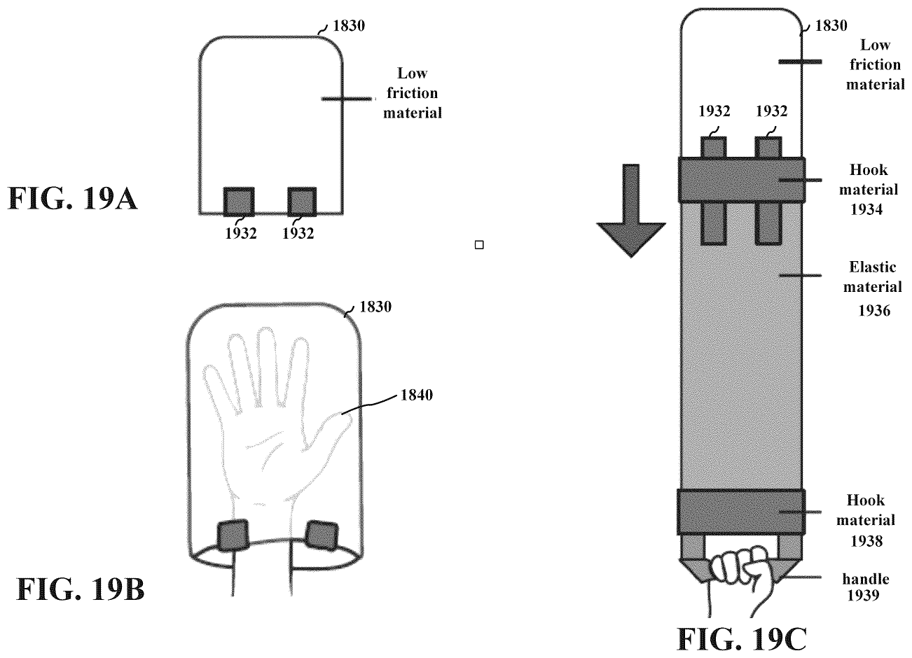

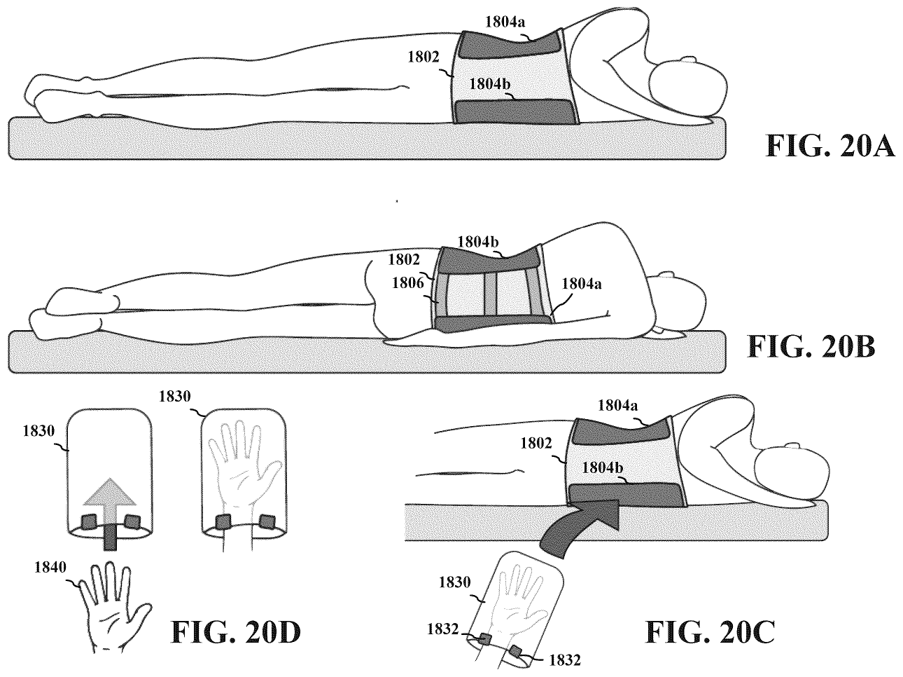

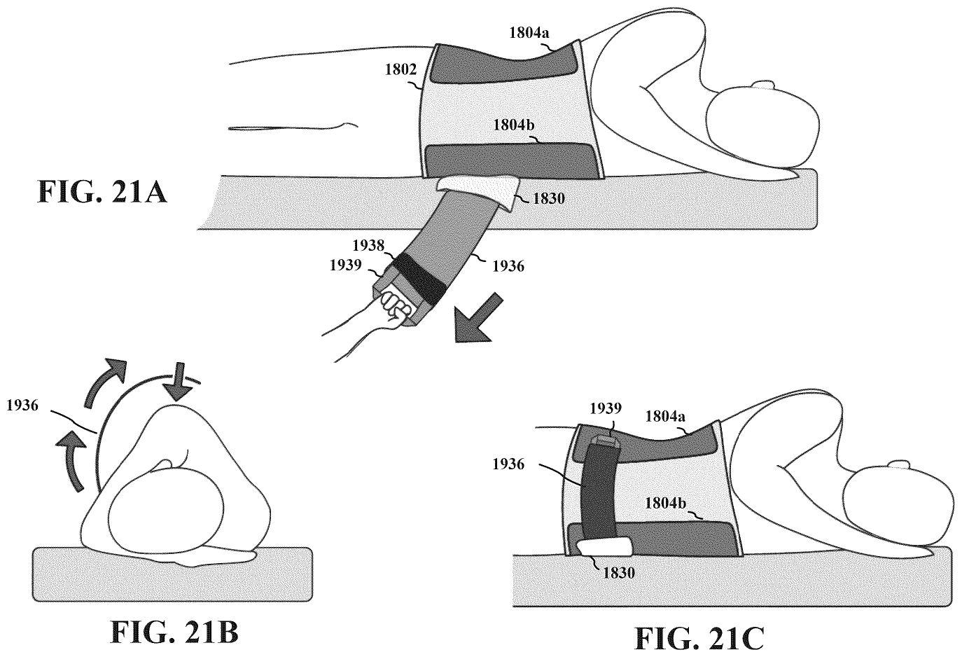

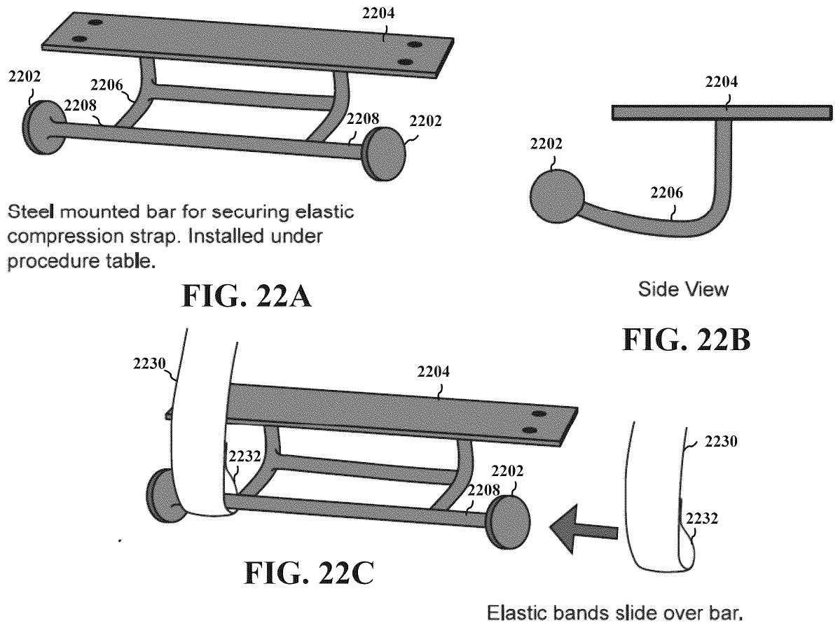

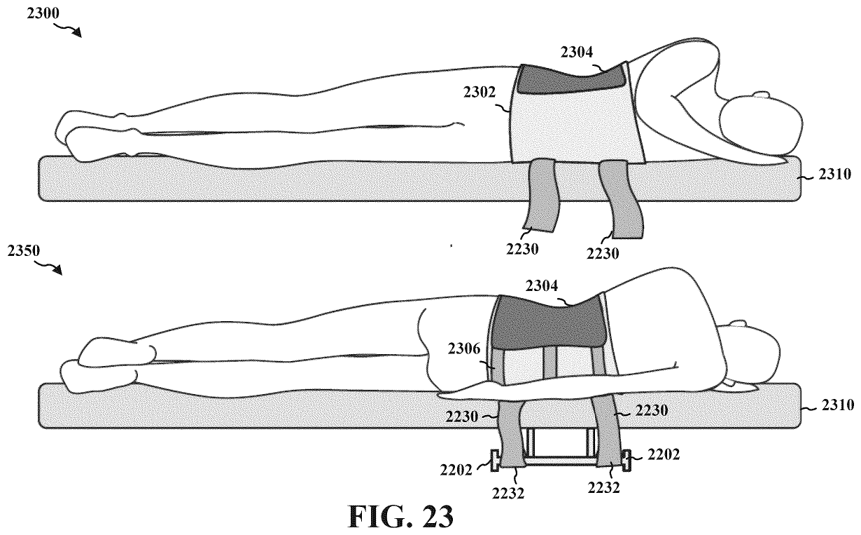

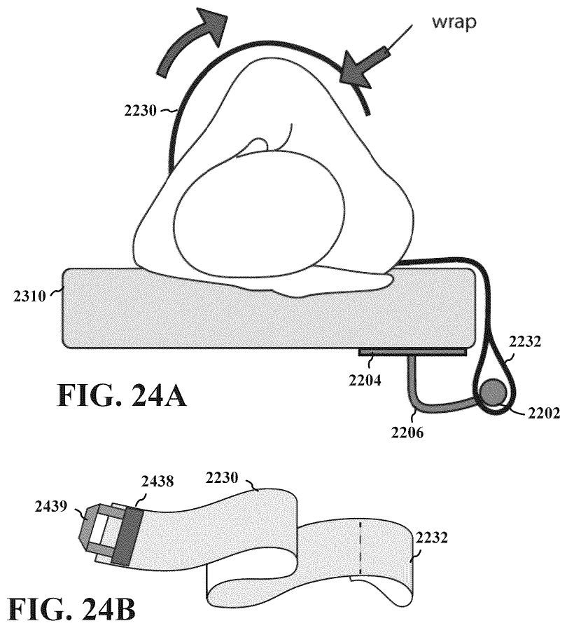

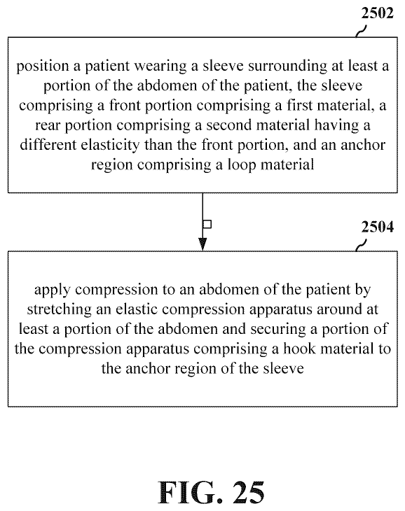

FIG. 16B, FIG. 17B, FIG. 17C, and FIG. 17D illustrate examples aspects of a hook, or attachment piece, that may be provided as part of a compression apparatus, in accordance with aspects presented herein.

FIG. 18A and FIG. 18B illustrate examples of an endoscopy garment that can be worn by a patient and used to secure a compression apparatus, such as the compression apparatus illustrated in FIGS. 18C-21C, in accordance with aspects presented herein.

FIG. 18C, FIG. 19A, FIG. 19B, FIG. 19C illustrate example aspects of a compression apparatus having a low friction cover that provides a push and pull mechanism for applying the compression apparatus to a patient wearing an endoscopy garment, in accordance with aspects presented herein.

FIG. 20A, FIG. 20B, FIG. 20C, FIG. 20D, FIG. 21A, FIG. 21B, and FIG. 21C illustrate an example of a compression apparatus having a push and pull mechanism and being applied to a patient wearing an endoscopy garment, in accordance with aspects presented herein.

FIG. 22A, FIG. 22B, and FIG. 22C illustrate examples aspects of a compression apparatus that is configured to attach to a procedure table, in accordance with aspects presented herein.

FIG. 23 illustrates an example of a patient positioned on a procedure table having a compression apparatus attached to a bar at the table and positioned between the table and the patient, in accordance with aspects presented herein.

FIG. 24A illustrates an example of a compression apparatus securing to a procedure table with a bar, in accordance with aspects presented herein.

FIG. 24B illustrates an example of a compression apparatus that includes a band configured to attach to a procedure table, in accordance with aspects presented herein.

FIG. 25 illustrates an example flowchart showing aspects of a method of applying compression with a compression apparatus during an endoscopy procedure, in accordance with aspects presented herein.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

Aspects presented herein comprise a sleeve that surrounds the abdomen of the patient and a first anchor region that receives a first end of at least one compression apparatus that allows the at least one compression apparatus to extend across the sleeve from the first anchor region to the second anchor region such that a second end of the at least one compression apparatus is coupled to a second portion of the sleeve to provide a localized pressure or force to colon or small bowel. The sleeve may be placed on, e.g., worn by, a patient, or be placed under a patient on the bed, prior to a procedure being started, and enables external compression apparatuses requiring circumferential leverage (e.g. continuity around the abdomen and/or trunk of the patient) to function optimally to be applied intra-procedurally to assist in insertion, visualization, and/or withdrawal of an endoscope. The use of the sleeve enables constituent, targeted compression to be readily applied via a compression device even to a sedated patient, when it may be challenging to move the patient. The sleeve may be comprised in an endoscopy garment worn by the patient. The endoscopy garment may provide the anchor points external compression apparatus and may provide some level of coverage for the patient, while also being structured for the endoscopy procedure.

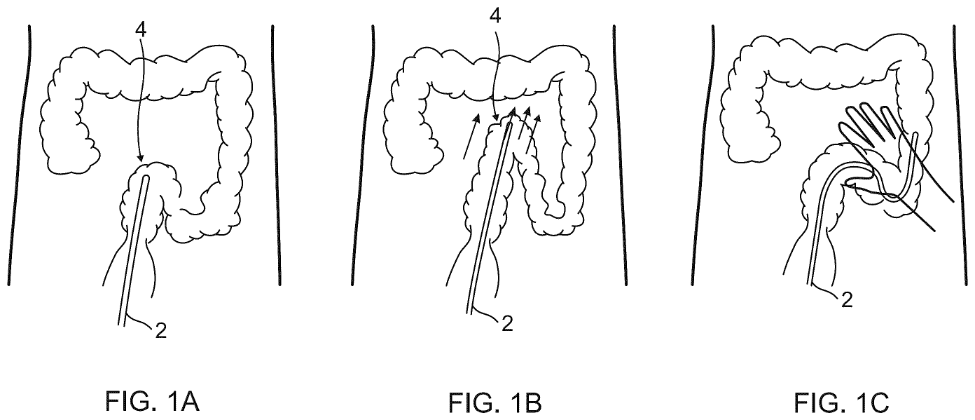

FIGS. 1A-1C, illustrate a sequence of steps of a colonoscopy, as one example of an endoscopy procedure. In FIG. 1A, a colonoscope 2 is inserted into the patient's rectum and advanced forward through the length of the colon. As the operator passes the colonoscope through the sigmoid region of the colon 4, the colonoscope may become impinged and cause distention and looping of the anatomy, as shown in FIG. 1B. The distention causes discomfort to the patient and increases the time required for the colonoscopy. In order to reduce the distended or looped area, a technician may apply manual pressure to abdomen of the patient. Among other examples, the technician may be a nurse, assistant, or other staff member. For example, the pressure may be applied by a nurse or surgical assistant as shown in FIG. 1C.

The application of manual pressure is time-consuming and places a physical toll on the technician. The effectiveness of the manual pressure varies depending on the particular technician's strength, technique, endurance, and training. In order to apply differential pressure and change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure may lead to injury of the patient or of the technician.

Many patients undergo colonoscopy while placed in the left lateral decubitus position on the stretcher or operating table. Additional information about the use of such manual pressure can be found in Prechel J A, Hucke R. Safe and effective abdominal pressure during colonoscopy: forearm versus open hand technique. Gastroenterol Nurs 2009; 32:27-30; quiz 31-2, the entire contents of which are incorporated herein by reference. In applying manual pressure, the technician may reach over the patient from the opposite side of the table and to deploy pressure by placing their hands against the patient's sigmoid colon and then leaning backwards, using their bodyweight for leverage to exert force. While these methods are generally effective at generating pressure, they have also been identified as a causative factor for the high rate of work-related injuries among endoscopy nurses and staff. Physicians performing colonoscopy suffer work-related musculoskeletal injury at a particularly high-rate as well. The most frequent site of physician injury is the right upper extremity which experiences peak torque forces when while operators are attempting to advance the scope through (a looping) sigmoid colon. Additional details can be found in Spanarkel M, Hathorn J P. Looping During Colonoscopy: A Major, Implied Cause of Injury Among Endoscopy Healthcare Providers and a Proposed Solution, 2013, the entire contents of which are incorporated herein by reference.

Aspects described herein may similarly be applied for other endoscopic procedures such as, but not limited to, sigmoidoscopy and retrograde enteroscopy procedures. Sigmoidoscopy is an examination of only the lower part of the colon, from the anus to the descending colon. An endoscope is inserted into the lower part of the colon. Enteroscopy is an examination of the small bowel. During retrograde enteroscopy, an endoscope is inserted in the anus and passed through the colon and the cecum and into the small bowel. Successfully navigating the loop-prone sigmoid region is necessary to complete both sigmoidoscopy and retrograde enteroscopy and thus aspects described herein can be used to help facilitate colonoscopy, sigmoidoscopy, retrograde enteroscopy, and other endoscopic procedures.

The small bowel is part of the intestine between the stomach and the colon. The small bowel comprises three segments, the duodenum, the jejunum, and the ileum.

It may be difficult to advance an endoscope in order to visualize the patient's entire small bowel due to the length, tortuosity/redundancy of the small bowel.

Push enteroscopy is an antegrade technique in which a physician uses a particular endoscope that is introduced through the patient's mouth. The physician slowly advances the scope, pushing the scope using pressure, as far as possible in order to visual the small bowel. However, this only allows for visualization of a limited portion of the small bowel.

Techniques and technologies may attempt to allow for greater visualization. As examples, single balloon enteroscopy, double balloon enteroscopy, and spiral enteroscopy are designed to overcome the challenge of advancing the scope deep into the small bowel. The three techniques may be considered, or may be referred to as, ‘deep small bowel enteroscopy.’

Each of the techniques can be used via an antegrade approach or a retrograde approach. In an antegrade approach, the scope is introduced through the patient's mouth and is advanced through the stomach in order to visualize the small intestine. In a retrograde approach, the scope is introduced through the rectum and through the colon and cecum in order to visualize the small intestine.

The techniques involve iterative advancement of an endoscope and an endoscopic overtube both of which can be advanced/withdrawn independently of one another.

As an example of balloon assisted enteroscopy, a double balloon enteroscopy (DBE) system may include an endoscope with an 8.5 mm diameter and a 200 cm working length, a 145 cm soft overtube with an outer diameter of 12.2 mm, and a specifically designed pump (the overtube may be backloaded onto the scope, a soft latex balloon may be attached to the tip of the enteroscope. The balloon can be inflated and deflated using a pump through the air channel in the enteroscope. The flexible overtube also has a latex balloon at its tip that can be inflated and deflated.

The DBE can be performed either via the mouth (anterograde approach) or the anus (retrograde approach). The insertion route may be selected according to the estimated location of the suspected lesion(s). With an antegrade examination, the enteroscope may be advanced using repetitive cycles of balloon inflation/deflation and scope advancement. During one cycle of the procedure, the scope may be advanced until no further forward progress is made. The balloon on the tip of the scope may then be inflated, anchoring the small bowel. Once the small bowel is anchored by the enteroscope balloon, the balloon on the overtube may be deflated (having been inflated during the prior cycle) and advanced until it reaches the inflated balloon on the scope. The balloon at the end of the overtube may then be inflated, and with both balloons inflated, the scope and overtube may be gently withdrawn to remove loops and pleat the small bowel onto the overtube. The balloon on the scope may then be deflated, and the cycle may be repeated until the scope can no longer be advanced or the lesion of interest is reached. The procedure may be carried out in reverse during withdrawal of the scope.

An endoscope, as in a double balloon endoscopy procedure, may be inserted through the overtube, and the endoscope balloon may be inflated. The overtube may be advanced along the endoscope, and the overtube balloon may be inflated. The endoscope balloon may then be deflated, and the endoscope may be advanced farther into the intestine. The endoscope balloon may then be deflated. The overtube may be advanced along the endoscope, and the overtube may then be deflated. The endoscope and overtube may be pulled/moved/withdrawn in the opposite direction in order to straighten the path through the intestine. Then, the endoscope balloon may be deflated, and the endoscope may be advanced, again, farther into the intestine. The procedure may be repeated, or performed iteratively in order to allow visualization of different areas of the intestine.

The procedure may be similar, or the same, for retrograde examinations. In retrograde examinations, the cycles of the scope advancement and withdrawal may be carried out in the colon, as well as in the small bowel. The cycles of advancement may help to ensure that there is no colonic looping prior to intubation of the terminal ileum, in some aspects.

In a single balloon enteroscopy, a enteroscope having a 200 cm high-resolution enteroscope with a 2.8 mm working channel may be used, for example. As an example, the overtube may be 140 cm long with a 13.2 mm outer diameter. The overtube may be equipped with a silicone balloon at its tip, which can be inflated and deflated.

The technique for single balloon enteroscopy is similar to that used for double balloon enteroscopy. Like double balloon enteroscopy, single balloon enteroscopy may use an overtube with a distal balloon to aid with scope advancement through the small bowel. However, SBE uses the scope's flexible tip to anchor the scope, rather than a second balloon on the tip of the enteroscope. Anchoring stabilizes the scope, permitting advancement of the overtube. The overtube balloon is then inflated, and the tip of the enteroscope is straightened. With the overtube balloon inflated, the scope and overtube can be withdrawn, pleating the small bowel onto the overtube. The enteroscope is then advanced. Like double balloon enteroscopy, the cycle of advancement and withdrawal may be repeated until the scope can no longer be advanced or the lesion of interest is reached

Spiral enteroscopy is an alternative to balloon-assisted enteroscopy for evaluation of the small bowel.

Spiral enteroscopy may use an overtube with a soft raised helix at its distal end. As an example, a 118-cm overtube may be compatible with enteroscopes that are 200 cm long and between 9.1 and 9.5 mm in diameter (e.g., the enteroscopes used for double balloon or single balloon enteroscopy, but not standard enteroscopes). The spirals on the overtube are either 4.5 mm (low profile) or 5.5 mm (standard profile) high. The overtube has a coupling device that affixes it to the scope, while permitting rotation of the overtube. By rotating the overtube clockwise, the small bowel may be pleated onto the overtube, and the enteroscope is advanced.

As described herein, the sleeve, e.g., or garment comprising the sleeve, in combination with the one or more compression apparatuses enables compression to be applied and/or adjusted at various points of the different procedures, e.g., as the scope is iteratively advanced deeper into the small bowel. The different levels and mechanisms of compression, presence/absence of compression, and/or targeted areas of compression may assist with the advancement of the scope, withdrawal of the scope, and/or visualization of the small bowel, colon, etc.

Compression delivered with a sleeve and at least one external compression apparatuses including any of the aspects described in connection with FIGS. 1A-12B may also be employed for spiral enteroscopy. For spiral enteroscopy, forward pressure may be applied to the enteroscope when rotation is started to advance or insert the scope. In some aspects, irrigation using a water get may assist with lumen visualization and lubrication, and intermittent abdominal compression and release may be provided by the compression apparatus through application, release and adjustment. In some aspects, consistent compression may be applied through some aspects of the advancement and/or withdrawal of the scope. Spiral enteroscopy relies on passive pleating and unpleating of the bowel, e.g., without forceful advancement or withdrawal of the scope. With an antegrade approach, the insertion proceeds through the stomach. A forward pressure during intubation of the pylorus may generate a gastric loop that may be reduced by straightening the enteroscope. In some aspects, a compression apparatus applied to the sleeve may apply compression to assist in avoiding looping.

In an antegrade approach for spiral enteroscopy, the scope may be passed through the fixed ligament of Treitz. Deeper passage into the small bowel may be achieved with the lumen decompressed. In some aspects, compression may be applied or adjusted by an external compression apparatuses applied to the sleeve in order to assist insertion and/or advancement. Passage through the small bowel may be achieved with forward rotation of the spiral segment and gentle forward pressure on the enteroscope. The passage may be assisted by application or adjustment of the compression apparatus and its application upon the sleeve. In some aspects, sharply angled bends may be encountered, and abdominal compression may be applied, adjusted, or maintained over the area by the compression apparatus followed by gentle forward pressure on the enteroscope. The sequence of compression application/adjustment and forward pressure on the enteroscope may be repeated until the bend can be successfully traversed by the enteroscope.

In the retrograde approach for spiral enteroscopy, abdominal compression may be applied as needed, through application or adjustment of compression apparatus applied to the sleeve in order to assist the spiral segment to be advanced across the value into the terminal ileum. Compression may also be applied or adjusted while passing through the small bowel in order to avoid looping and/or to address looping that does occur. The compression may be applied or adjusted for the area, and then the forward rotation of the spiral segment may be resumed.

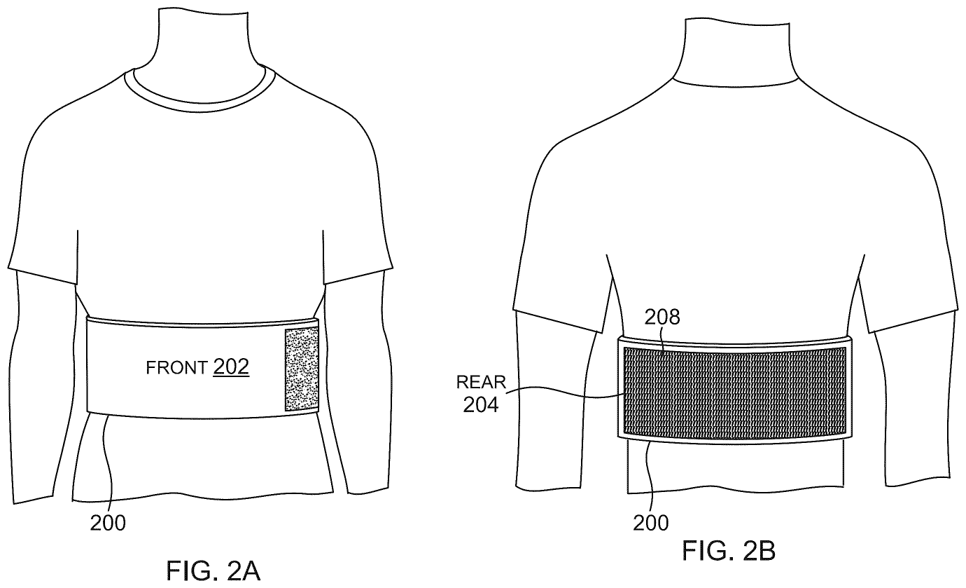

FIGS. 2A and 2B illustrate an example sleeve device. The sleeve 200 comprises a front portion 202, a rear portion 204, and one or more anchor region on at least the front portion or the rear portion. The sleeve 200 is configured to surround at least a portion of the abdomen of the patient during endoscopic procedures. The sleeve 200 acts as a modular base that may be worn by patients, or placed under patients, in preparation of and/or during an endoscopy procedure that may require application of external and/or localized pressure to assist or allow the endoscope, or similar instrument, to advance the instrument during the procedure. The sleeve 200 is a modular base that may be worn by patients that may act as an anchoring base for one or more compression apparatuses that may deliver the external and/or localized pressure as desired. The sleeve may receive a variety of compression apparatuses, bands or attachments that may have different purposes. In some aspects, the sleeve may include a unitary section that encircles the abdomen of the patient. In some aspects, the sleeve may be a portion of a more comprehensive endoscopy garment.

The front portion 202 of the sleeve, when worn by the patient, may cover an abdominal region of the patient, while the rear portion 204 may cover a lower back region of the patient. The front portion 202 may be comprised of a first material that may have elastic properties. The first material of the front portion may expand to accommodate various abdomen sizes of different patients to allow the sleeve to be utilized for patients of different body sizes. The rear portion 204 may be comprised of a second material that has less elasticity than that of the first material. In some aspects, the second material may be comprised of a semi-rigid or reinforced material. In some aspects, the second material may be comprised of one or more materials having different elasticities. For example, the second material may have a first elasticity along a rear central region of the rear portion, while the second material may have a second elasticity along the outer ends of the rear portion that are proximate the front portion of the sleeve. The first elasticity may be less elastic than the second elasticity, such that the second material having the first elasticity is maintained substantially aligned with a spine of the patient. The rear portion comprising the second material may maintain or align the rear portion of the sleeve onto a back region of the patient. The rear portion comprising the second material may be configured to provide a support structure to assist the one or more compression apparatuses to apply the pressure during the procedure. In some aspects, the rear portion may counter pressure applied by the one or more compression apparatuses to maintain or align the rear portion of the sleeve with the back region of the patient.

The sleeve 200 may comprise the one or more anchor regions 208 on the front portion 202 and/or the rear portion 204. In the example of FIG. 2B, the rear portion 204 comprises at least a second anchor region 208 that covers a substantial portion of the rear portion 204. However, in some aspects, the rear portion 204 may comprise a plurality of anchor regions 208 instead of the second anchor region that substantially covers the rear portion 204. The front portion 202 may comprise the one or more anchor regions 208 in a manner similar to the rear portion 204. The front portion 202 may comprise a first anchor region along a left region of the sleeve. The one or more anchor regions 208 may be configured to receive one or more compression apparatuses in order to apply the pressure during the procedure.

In some aspects, the first anchor region may be proximate or adjacent the front portion 202 and/or the rear portion 204. For example, the first anchor region may be on the front portion proximate the rear portion, such that the first anchor region covers at least a part of the front portion. In another example, the first anchor region may be on part of both the front portion and the rear portion, such that the first anchor region covers at least a part of both the front portion and the rear portion. In another example, the first anchor region may be on the rear portion proximate the front portion, such that the first anchor region covers at least a part of the rear portion. The first anchor region may be configured to removably couple with an end of the one or more compression apparatuses. The one or more compression apparatuses, when applied over the abdomen of the patient between the first anchor region and at least the second anchor region, may apply pressure onto the abdominal region of the patient during the procedure.

In some aspects, the rear portion of the sleeve may comprise a second anchor region that covers substantially all of the rear portion. In such instances, the second anchor region may provide a surface to receive the second end of the compression apparatus that covers a substantial part of the rear portion.

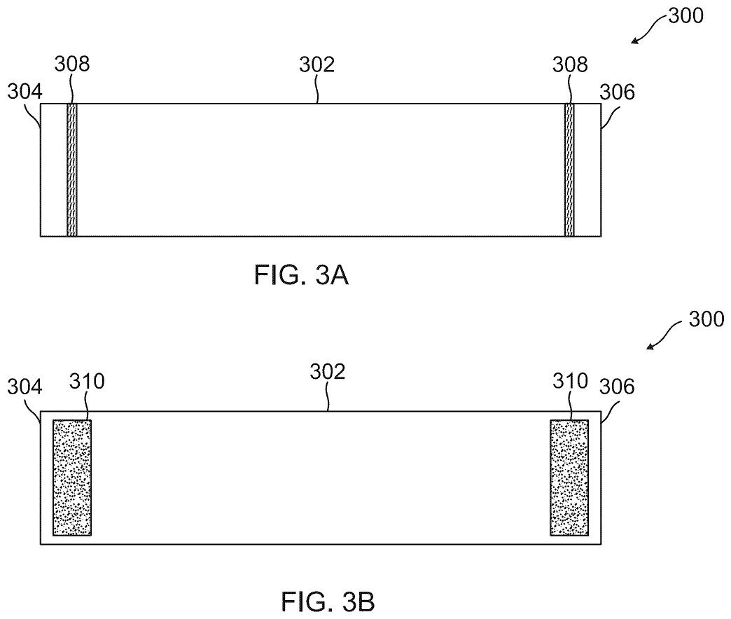

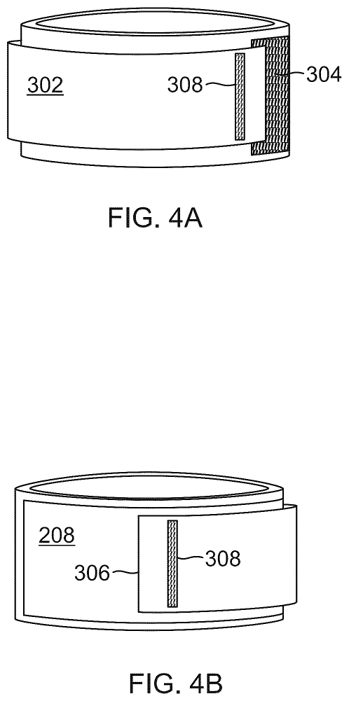

FIGS. 3A and 3B illustrate an example of a compression apparatus 300. The compression apparatus 300 comprises a first end 304, a body 302, and a second end 306. The first end 304 may be configured to be removably coupled to the first anchor region, as shown for example in FIG. 4A. The second end 306 may be configured to be removably coupled to at least the second anchor region of the sleeve, such that the compression apparatus 300 may extend over the abdomen of the patient and apply the pressure upon at least a portion of the abdomen of the patient, as shown for example in FIGS. 4A and 4B.

The first end 304 of the compression apparatus 300 may comprise a fastener 310 such that the first end may couple to the first anchor region of the sleeve 200. For example, the fastener 310 may comprise a plurality of hooks while the first anchor region comprises a plurality of loops, such that the plurality of hooks of the fastener 310 of the first end 304 may couple with the first anchor region. In some aspects, the fastener 310 may comprise a plurality of loops while the first anchor region comprises a plurality of hooks, such that the first end may couple with the first anchor region. The fastener 310 may comprise many different fastening devices and is not intended to be limited to a hook or loop configuration. The fastener 310 is configured to correspond with the first anchor region of the sleeve 200 such that the first end 304 may be removably coupled to the first anchor region.

The second end 306 of the compression apparatus 300 may also comprise a fastener 310 configured in a similar manner as fastener 310 of the first end 304. The second end 306 of the compression apparatus 300 may be removably coupled to the one or more anchor regions 208. In some aspects, the second end of the compression apparatus may be removably coupled to at least the second anchor region. The body 302 of the compression apparatus 300 may extend over a portion of the abdomen of the patient and over part of the front portion of the sleeve, such that when the first end 304 is coupled to the first anchor region and the second end 306 is coupled to the one or more anchor regions 208 (e.g., at least the second anchor region) while the body 302 applies pressure on the portion of the abdomen of the patient.

In some aspects, the first end 304 or the second end 306 of the compression apparatus 300 may each comprise a handle 308. The handle 308 may be on the band opposite the fastener 310. The handle 308 at the first end 304 may be configured to assist with the placement or positioning of the first end 304 with the first anchor region. The handle 308 at the second end 306 may be configured to assist with the placement or positioning of the second end 306 with at least the second anchor region. For example, in some aspects, the handle 308 may provide an opening or a loop such that the opening or the loop may be used to position or place the first end onto the first anchor region. In some aspects, the handle 308 may be used to position or place the second end onto at least the second anchor region. At least one advantage of the handle 308 is that at least the first end of the compression apparatus may be coupled to the first anchor region when the patient is laying down, such that access to the sleeve and/or the first or second anchor region may be limited or moving the patient is difficult due to the patient being sedated or unable to move.

Although the example of the compression apparatus 300 of FIGS. 3A and 3B is depicted as a strap or elastic band, the disclosure is not intended to be limited to the aspects presented herein. The compression apparatus may be in many different forms and is not intended to be limited to a strap or elastic band. For example, in some aspects, the compression apparatus may comprise a shaped insert (e.g., gel, foam, silicone, or the like) with an elastic compression band that may be removably coupled to one or more anchor regions, and may be configured to pull the insert into the abdomen of the patient to provide targeted and/or directionally-oriented pressure. In some aspects, the compression apparatus may comprise a pressure pillow/cushion (e.g., gel, foam, silicone, or the like) with elastic or non-elastic bands removably coupled to the one or more anchor regions, and may be configured to hold the pressure pillow in place against the abdomen of the patient based on the positioning of the patient (e.g., while the patient is rolled ‘on top of pillow’), with patient body weight/gravity pushing pillow into abdomen to provide pressure. In some aspects, the compression apparatus may comprise one or more inflatable bladders that may be configured to inflate to provide targeted compression once attached and pulled against body. In some aspects, the compression apparatus may comprise one or more mechanical pressure applicators that may be configured to extend outward (e.g., inward into the abdomen) in a piston-like fashion to apply targeted pressure during a colonoscopy. In some aspects, the compression apparatus may comprise a combination of any of the examples discussed herein.

In some aspects, the sleeve may be comprised as a unitary sleeve. The unitary sleeve may be a modular base garment (e.g., endoscopy garment) that is worn by the patient and is an anchoring base for attachments (e.g., compression apparatuses) that deliver compression when needed. The sleeve may allow for patients to wear the modular base garment in anticipation of determining whether localized pressure is needed during the procedure. The modular base garment may be part of a system that may operate independently of user input. For example, the system may be configured to sense/receive data and feedback regarding the positioning of the scope and if looping is occurring. In instances where looping has occurred or advancement of the scope is hindered/prevented, the system may be configured to determine and deliver an optimal type of compression to an optimal location to minimize looping and/or to allow advancement of the scope. At least one advantage of the disclosure is that modular garment may enable the compression apparatuses to be re-usable.

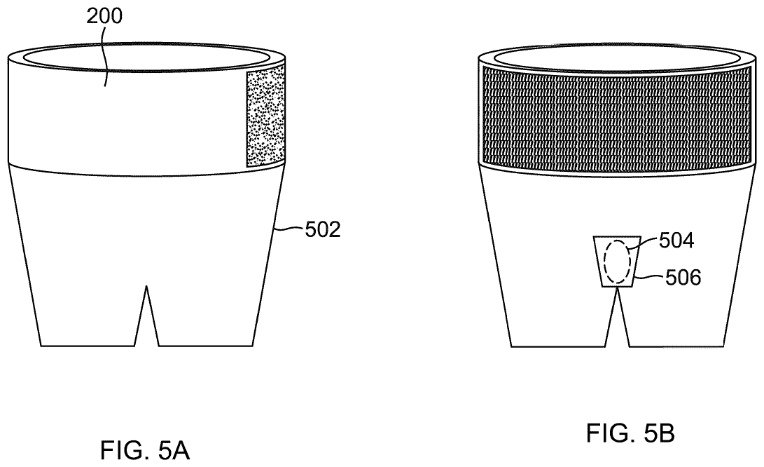

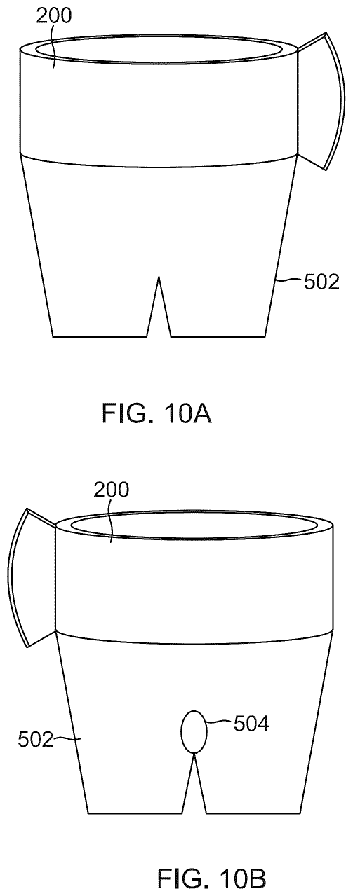

In some aspects, the garment may be configured to provide access to a rectum of the patient while covering the lower portion of the body of the patient. The garment may extend around a groin region of the patient, such that the garment extends around the groin region and may provide support for the sleeve to receive the one or more compression apparatuses, as shown for example in FIGS. 5A, 5B, 10A, and 10B. The garment extending around the groin region may assist to hold the sleeve around the abdominal region of the patient while the compression apparatuses are applied to the sleeve. The garment may comprise a lower portion 502 that is coupled to a lower end of the sleeve 200. The lower portion 502 may have openings for a respective leg of the patient to allow the patient to wear the garment. For example, the lower portion of the garment may have a pant-like shape that wraps around each leg of the patient. The garment may be form fitting about the legs of the patient. The rear opening 504 provides access to the rectum of the patient while concealing at least the groin area of the patient. The rear opening may comprise a removable panel 506 that may be fastened to the sleeve to conceal the rear opening and unfastened to provide access to the rectum of the patient via the rear opening. At least one advantage of the disclosure is that the garment may cover the patient to reduce or minimize exposure of the body of the patient during such procedures, while providing structural support for the one or more compressions bands that may be used during such procedures.

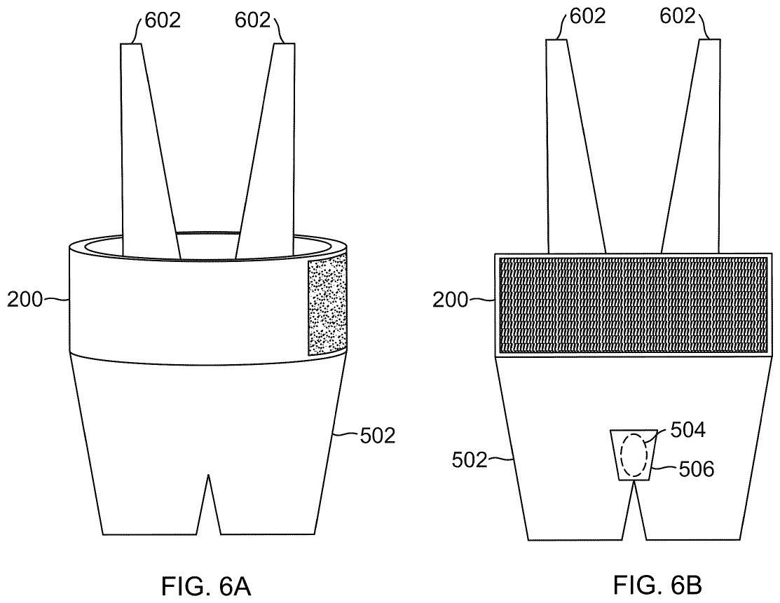

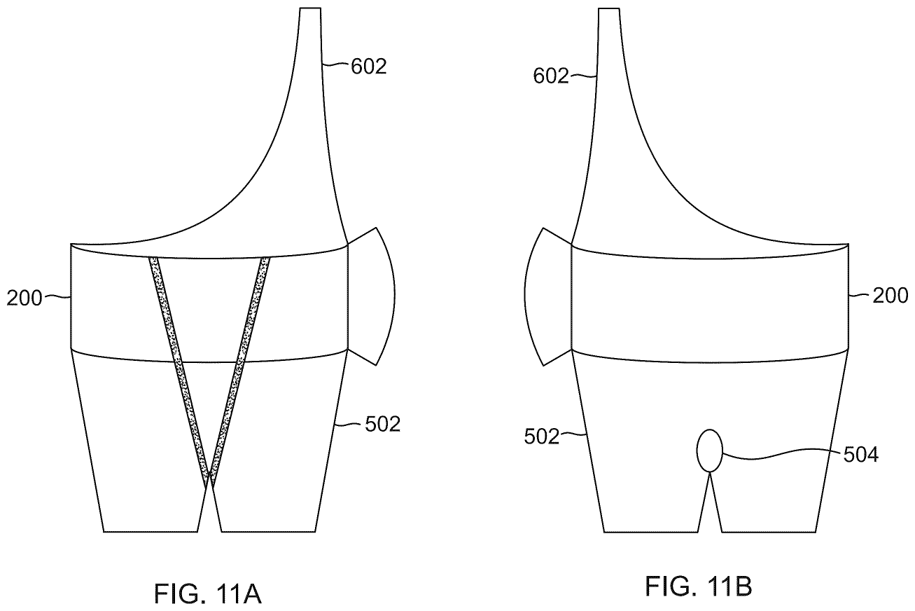

In some aspects, as shown for example in FIGS. 6A, 6B, 11A, and 11B, the garment may further comprise one or more shoulder straps 602. The one or more shoulder straps 602 may wrap around a shoulder region of the patient. The garment wrapped around the shoulder region of the patient may provide support for sleeve to receive the one or more compression bands. The one or more shoulder straps may assist to hold the sleeve around the abdominal region of the patient when the compression apparatuses are applied to the sleeve. The one or more shoulder straps may counter pressure applied by the one or more compression apparatuses to maintain or align the rear portion of the sleeve with the back region of the patient.

In some aspects, as shown for example in FIGS. 11A and 11B, the garment comprises one shoulder strap 602. The one shoulder strap 602 wraps around a shoulder of the patient while minimally covering the chest or torso region of the patient, which allows for the placement of any sensor pads that may be placed on the patient during the procedure. In some aspects, the garment may comprise one or more release strips 510 that run along a front region of the garment. The one or more release strips 510 may comprise fastening devices that allow the garment to be removed from the patient without having to disrobe or get undressed. The fastening devices may comprise hook and loop materials, buttons, zippers, or the like. The one or more release strips 510 may allow the garment to be easily removed from the patient. For example, in the event of an emergency, anything covering the patient should be able to be quickly removed. In another example, given the nature of colonoscopy procedures, there is a potential for the garment to become soiled during the procedure, and should this occur, it would be ideal for the garment to be removed and disposed of with any other contaminated consumables, instead of sending the patient out to the recovery area with the soiled garment on, and having the patient change out of soiled garment themselves. At least one advantage of the release strips 510 is that the construction and placement of the strips (e.g., near an umbilicus region) would not result in interference with the application of any compression apparatus or bands nor would the release strips be subjected to any increased tensile strain when the compression apparatus is applied, due in part to the compression apparatus pulling or imparting a force against or onto the rear portion of the sleeve.

The sleeve may be configured to comprise the one or more anchor regions in different configuration. In some aspects, for example, the first anchor region may extend along at least a portion of the sleeve, such as, but not limited to, a left region of the sleeve. For example, in some aspects, the first anchor region may extend along the entire width of the sleeve 200, while in some aspects, the first anchor region extends along part of the width of the sleeve. Each of the one or more anchor regions may provide a surface to receive the first end or the second end of the one or more compression apparatuses. For example, the first anchor region may provide a surface to receive the first end of the one or more compression bands. The first anchor region may be on a left side of the sleeve, such that the first anchor region is arranged to be proximate, adjacent, or aligned with a left hip region of the patient. The first anchor region being near the left hip region of the patient allows for the compression band to apply the pressure onto the abdominal region of the patient while the patient is laying on their left side during the procedure.

In some aspects, the surface of the first anchor region may comprise a fastener that corresponds with a fastener on the first end of the compression apparatus, in order to removably couple the compression apparatus to the first anchor region. In some aspects, the first anchor region may comprise a plurality of anchor regions that collectively extend along at least a portion of the sleeve.

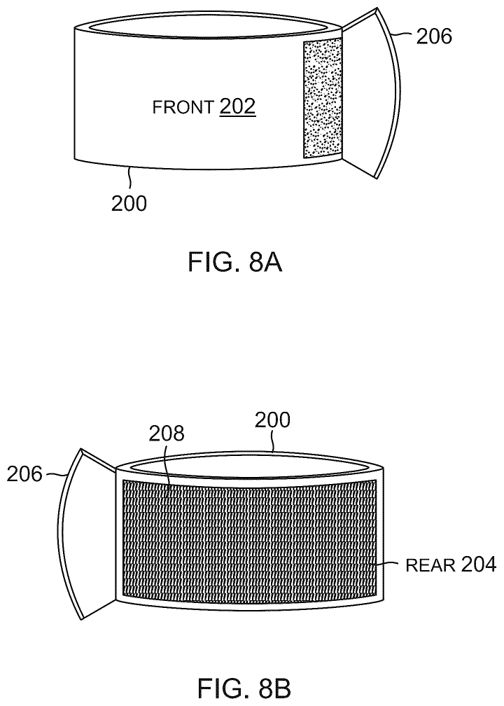

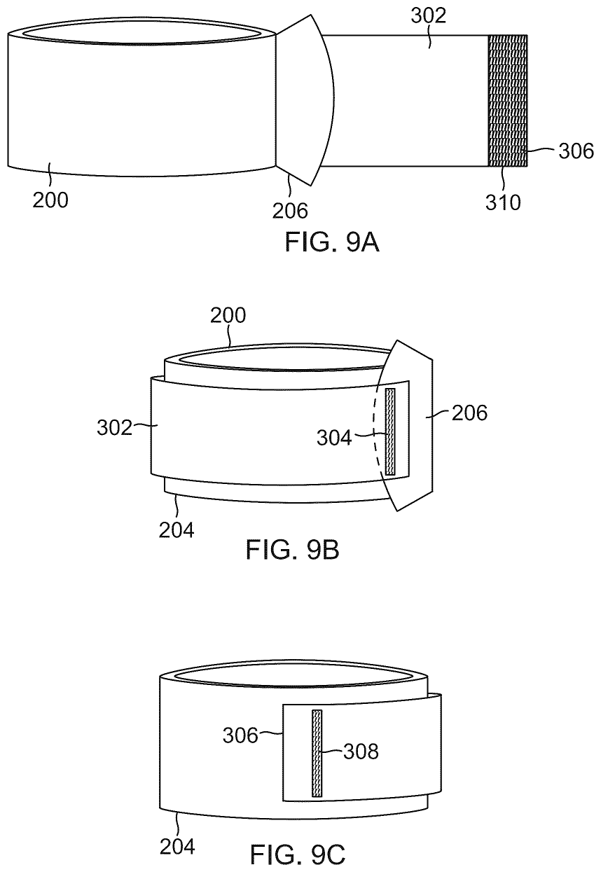

In some aspects, as shown for example in FIGS. 8A and 8B, the sleeve 200 may further comprise at least one attachment extension 206. The at least one attachment extension 206 may be proximate or adjacent the front portion 202 and/or the rear portion 204. The at least one attachment extension 206 may be configured to removably couple with an end of the one or more compression apparatuses. The one or more compression apparatuses, when stretched over the front portion 202 and between the at least one attachment extension and the one or more anchor regions, may be configured to apply pressure onto the abdominal region of the patient during the procedure, as shown for example in FIGS. 9A-9C.

The first end 304 of the compression apparatus 300 may comprise the fastener 310 such that the first end may couple to the at least one attachment extension 206 of the sleeve 200. For example, the fastener 310 may comprise a plurality of hooks while the at least one attachment extension 206 comprises a plurality of loops, such that the plurality of hooks of the fastener 310 of the first end 304 may couple with the at least one attachment extension 206. In some aspects, the fastener 310 may comprise a plurality of loops while the at least one attachment extension 206 comprises a plurality of hooks, such that the first end may couple with the at least one attachment extension. The fastener 310 may comprise many different fastening devices and is not intended to be limited to a hook or loop configuration. The fastener 310 is configured to correspond with the attachment extension 206 of the sleeve 200 such that the first end 304 may be removably coupled to the attachment extension 206.

The second end 306 of the compression apparatus 300 may also comprise a fastener 310 configured in a similar manner as fastener 310 of the first end 304. The second end 306 of the compression apparatus 300 may be removably coupled to the one or more anchor regions 208. The body 302 of the compression apparatus 300 may extend over a portion of the abdomen of the patient, such that when the first end 304 is coupled to the attachment extension 206 and the second end 306 is coupled to the one or more anchor regions 208 the body 302 applies pressure on the portion of the abdomen of the patient.

In some aspects, the first end 304 or the second end 306 of the compression apparatus 300 may each comprise a handle 308. The handle 308 may be on the band opposite the fastener 310. The handle 308 at the first end 304 may be configured to assist with the placement or positioning of the first end 304 with the attachment extension 206. The handle 308 at the second end 306 may be configured to assist with the placement or positioning of the second end 306 with the anchor region 208. For example, in some aspects, the handle 308 may provide an opening or a loop such that the opening or the loop may be used to position or place the first end onto the attachment extension. In some aspects, the handle 308 may be used to position or place the second end onto the anchor region. At least one advantage of the handle 308 is that at least the first end of the compression apparatus may be coupled to the attachment extension when the patient is laying down, such that access may be limited or moving the patient is difficult due to the patient being sedated or unable to move.

In some aspects, the at least one attachment extension 206 may extend along at least a portion of the sleeve. For example, in some aspects, the at least one attachment extension may extend along the entire width of the sleeve 200, while in some aspects, the at least one attachment extension extends along part of the width of the sleeve. The at least one attachment extension may provide a surface to receive the first end of the one or more compression apparatuses. The surface of the at least one attachment extensions may extend from the sleeve such that the first end of the one or more compression apparatuses may be removably coupled to the surface of the at least one attachment extension. The at least one attachment extension may be on a left side of the sleeve, such that the at least one attachment extension is arranged to be proximate, adjacent, or aligned with a left hip region of the patient. The attachment extension being near the left hip region of the patient allows for the compression apparatus to apply the pressure onto the abdominal region of the patient while the patient is laying on their left side during the procedure.

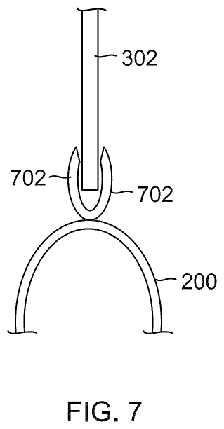

In some aspects, the surface of the at least one attachment extension may comprise a fastener that corresponds with a fastener on the first end of the compression apparatuses, in order to removably couple the compression apparatus to the at least one attachment extension. In some aspects, the at least one attachment extension may comprise a plurality of attachment extensions that extend along at least a portion of the sleeve, as shown for example in FIG. 7. In the example of FIG. 7, the sleeve 200 comprises two attachment extensions 702 that form a v-shaped groove, wherein the first end of the compression apparatus 300 is received by the v-shaped groove to removably couple the first end of the compression apparatus 300 to the plurality of attachment extensions that form the v-shaped groove. The dimensions of the plurality of attachment extensions that for the v-shaped groove may be the same or different.

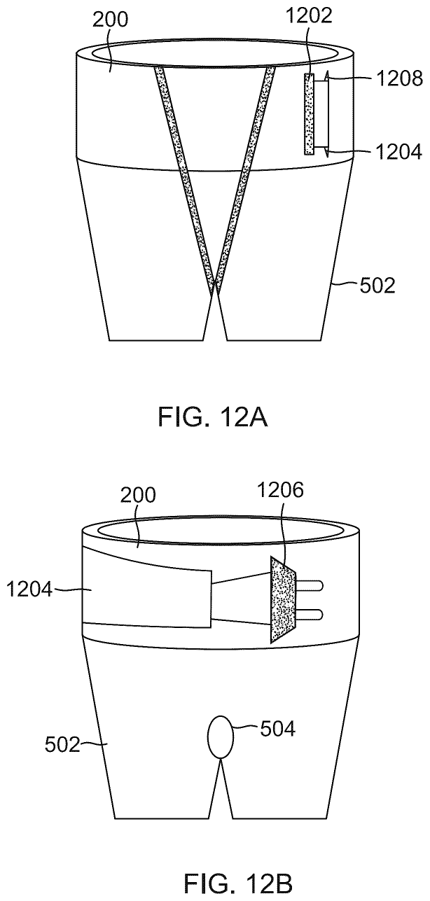

In some aspects, as shown for example in FIGS. 12A and 12B, the garment may comprise an anchor region 1202 that may be displaced along part of the front region of the sleeve. The garment may comprise an independently moving component that extends around a left side of the sleeve from the front region to the back region. The moving component comprises a strap 1208 comprising an anchor region 1202 and a closure mechanism 1206 opposite the anchor region 1202. The closure mechanism 1206 may be removably coupled to the anchor region of the rear portion. In some aspects, the strap 1208 of the moving component may pass through a tunnel or loop 1204 that may be on a left side of the sleeve. The tunnel or loop may be interiorly lined with very low friction medical fabrics. The strap of the moving component may also be comprised of the very low friction fabric as well.

A compression apparatus (e.g., compression band 300) may be removably coupled to the anchor region 1202. The compression apparatus may connect with the anchor region 1202 on an end of the strap 1208. The compression apparatus may then be pulled tight under the patient by pulling the other end (e.g., 1206) of the strap tightly and fastening to an anchor section of the rear portion of the sleeve. In some aspects, the opposite end of the compression apparatus may be pulled tight and removably coupled to the anchor region of the rear portion of the sleeve. The compression apparatus may be pulled and/or extended at each of the opposing ends in an iterative manner to achieve desired compression.

Improving patient comfort and reducing complications, both during and following endoscopic procedures is very important. Aspects presented herein reduce patient discomfort and complications by helping to prevent and reduce sigmoid looping, which can be a primary cause of patient pain and discomfort.

Patients often become cold or uncomfortably chilled once they change into the garments such as hospital gowns typically worn while undergoing an endoscopic procedure. Many patients request and are provided with blankets, and some facilities provide electric heated blankets, or blankets that have been previously warmed. The heated blankets provide physical warmth, but also the warm blanket tends to relax the patient and relieve anxiety or discomfort the patient may be experiencing. Aspects presented herein may also help the patient to feel warm and comfortable during the procedure. For example, the use of an endoscopy garment may provide added warmth for the patient, and may facilitate a more relaxed mindset through added coverage, while providing anchor points that can be used to couple with a compression component and while providing an open region for the endoscopy procedure.

To additionally enhance patient comfort, certain aspects of the invention are designed to be single-use, and to remain fastened in place on the patient during the procedure and/or following the procedure. For example, maintaining the compression applied by the device during the withdrawal phase of the procedure and while imaging is performed may help improve the detection of adenoma. The device may be maintained on the patient to reduce the common post-procedure complications of bloating and abdominal pain caused by bloating. Otherwise known as gaseous distention, bloating occurs following endoscopy procedures because physicians often use compressed air or carbon dioxide to insufflate parts of the bowel that are difficult to see and examine. The gas opens up the area to allow for a more complete visualization, enhancing the efficacy of the procedure. However, the gas also remains in the patient until it is either absorbed or expelled. Expulsion is the primary gas removal mechanism as absorption is a very inefficient process. Gaseous distention is a primary post-procedure complication and a frequent complaint from patients. However, when the wrap described herein remains in place after the procedure, the lower abdominal compression generated by the device allows the bowel to more rapidly evacuate trapped by directing excess gas towards the rectum. As a result, the severity and duration of post-procedure bloating and associated abdominal pain may be reduced.

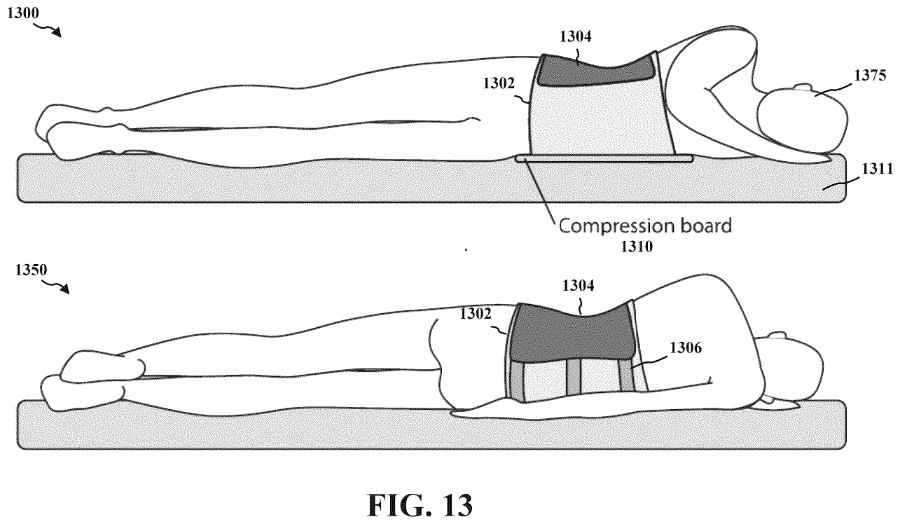

Various aspects or mechanisms may be included to increase leverage and/or anchoring for applying compression using an endoscopy garment as described herein. The example aspects and mechanisms also enable the compression to be applied and/or adjusted during an endoscopy procedure, in which the patient may be sedated and lying on their side. FIGS. 13-17D illustrate example aspects of a compression board that may be used in connection with the endoscopy garment and a removable compression apparatus (e.g., similar to 300), which may also be referred to as a compression band, a band, a compression strap, a strap, an elastic strap, a wrap, a compression wrap, an elastic wrap, among other examples.

FIG. 13 illustrates a front view 1300 of a patient wearing an endoscopy garment 1302 having an anchor region 1304 for removably coupling a compression apparatus. The anchor region 1304 may correspond to 304 described in connection with FIG. 3, for example. FIG. 13 also shows a rear view 1350 of the patient wearing the endoscopy garment 1302 and showing that the garment may include one or more reinforcement sections, e.g., which may include sections of a material that has less elasticity than other portions of the garment. The reinforcement sections 1306 may assist in the application of compression to the abdomen of the patient. The front view 1300 shows a compression board 1310 (which may also be referred to as a compression surface) positioned between the patient 1375 and the procedure table 1311.

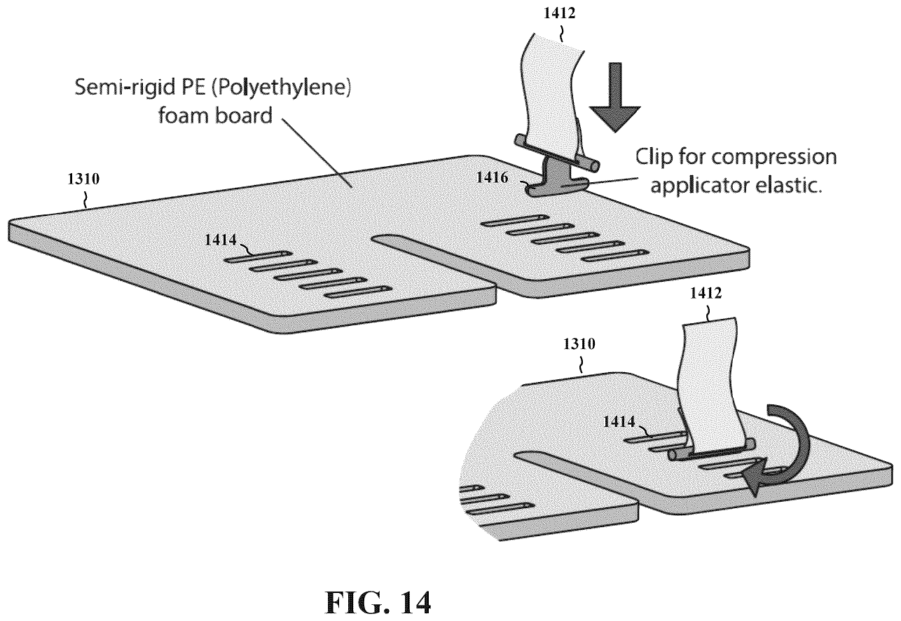

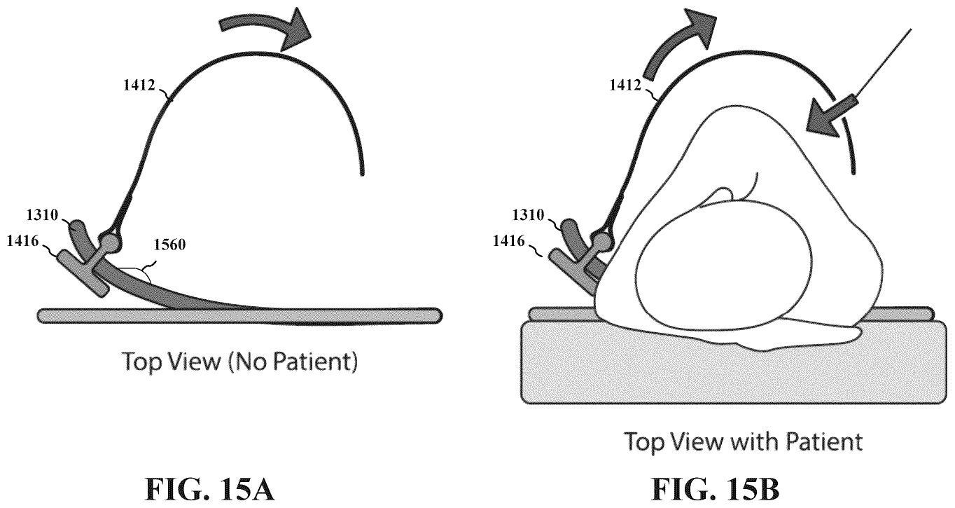

FIG. 14 illustrates that the compression board 1310 may include one or more attachment points that enable the compression apparatus to be coupled to the compression board 1310 and then pulled around the abdomen of the patient 1375 in order to apply targeted compression during an endoscopy procedure. As shown in FIG. 14, the compression board 1310 may a semi-rigid material. In some examples, the compression board may comprise a foam material, such as a semi-rigid Polyethylene foam board. The board 1310 may be placed on the table 1311 prior to patient arrival. The patient 1375 is then positioned on top of the board, e.g., with the board between the table 1311 and the patient 1375. If additional compression is preferred during an endoscopy procedure, a compression apparatus 1412 (e.g., compression band) may be coupled or attached to the board 1310, such as with a board hook 1416. FIG. 14 illustrates an example in which the board 1310 may include one or more openings 1414 that are configured to receive a hook 1416, which may also be referred to as a latch or clip, positioned at one end of the compression apparatus 1412, For example, a user can push the hook 1416 into an accessible slot nearest the patient's body and twist the hook relative to the opening 1414 to secure the compression apparatus to the board 1310. The user may then pull the compression apparatus (e.g. which includes an elastic band) over the patient 1375 and secures the compression apparatus to the anchor portion 1304. FIG. 15A illustrates an example of the compression apparatus 1412 attached to a board 1310 and pulled around an area in which the patient would be placed. In some aspects, the board may be shaped to assist in the application of pressure to the abdomen of the patient. In some aspects, the board may be rounded rather than flat. In some aspects, the board may include a shaped protrusion on the side toward the abdomen of the patient. In some aspects, and insert may be placed between the compression board and the abdomen of the patient. For example, FIG. 15A illustrates an example protrusion or insert 1560. When the board is pulled against the patient by the compression apparatus, the insert or protrusion from the surface of the board may apply a targeted compression to a particular portion of the patient's abdomen. FIG. 15B illustrates a view showing the compression apparatus 1412 coupled to the board 1310 and pulled around the patient 1375 to apply compression to the abdomen. In some aspects, the anchor portion may include a loop material that is configured to receive and hold a hook material at an end of the compression apparatus 1412. As an example, the hook and loop materials may include Velcro®. FIG. 16A shows a view of an example compression board 1310. FIG. 16B illustrates an example hook 1416 that may be positioned, secured, at an end of the compression apparatus 1412. FIG. 17A illustrates a full view of an example compression apparatus 1412 (e.g., compression band) that includes the hook 1416 and a hook material 1418 that is configured to attach to the anchor portion 1304 of the endoscopy garment. FIG. 17B and FIG. 17C illustrate side views of the hook 1416. FIG. 17D illustrates a top view of the hook 1416. In some examples, the hook may include a polypropylene or ABS injection molded clip/latch. In some aspects, the compression apparatus may include a knit elastic band and/or neoprene material.

FIG. 18A and FIG. 18B illustrate an example endoscopy garment 1802 that includes two anchor portions 1804a and 1804b for attaching a compression apparatus. In some aspects, the anchor portions may be referred to as loop patches provided on the left and right sides of the endoscopy garment, which wraps around the patient's abdomen. FIG. 18A illustrates a front view of the endoscopy garment, e.g. which would cover an abdomen of the patient. FIG. 18B illustrates a back view of the endoscopy garment, which would cover the patient's back. FIG. 18B illustrates that the endoscopy garment may include one or more reinforcement portions 1806, e.g., that include a different fabric or material that is less elastic than the other portion(s) of the endoscopy garment 1802. FIG. 18C illustrates an attachment mechanism that may be included as a part of the compression apparatus that attaches to the endoscopy garment. FIG. 18C illustrates a cover portion 1830 that is configured to surround a user's hand 1840. The cover portion may be referred to by various different names, such as a glove portion, a push/pull portion, or a low friction portion among other examples. The outer surface of the cover portion 1830 may include a material such as satin, polyester, and/or nylon. The material may be selected to allow the user to push, slide, or insert the cover portion under the patient, e.g., between the patient and the procedure table. For example, the material may have a lower friction material than other portions of the compression apparatus.

In some aspects, before the procedure, the patient, or another user, may apply an abdominal wrap with anchor patches (e.g., with a hook and/or loop material) on the left and right sides. As needed, the user may use a one-time push-pull compression glove to apply additional compression during an endoscopy procedure.

FIG. 19A illustrates a view of the cover portion 1830. FIG. 19B illustrates a view of the cover portion with the user's hand inserted into an opening at one side of the cover. The other parts of the compression band may be housed within the cover portion 1830. For example, the elastic band 1936 may be within the cover portion 1830 and fixed, e.g., sewn to the closed end of the cover opposite the open end. FIG. 19C illustrates that the compression apparatus may include the cover 1830, an elastic material 1936, a handle 1939, two portions of hook material 1934 and 1938, and attachment material 1932.

FIG. 20A and FIG. 20B illustrate that a patient may be lying on their side for the endoscopy procedure. A patient may be sedated prior to the procedure, and the patient may put the endoscopy garment 1802 on prior to the sedation. Once the patient is wearing the endoscopy garment, the patient may be positioned on the procedure table and sedated. As illustrated in FIG. 20A and FIG. 20B, the patient may be lying on one of anchor portions 1804a or 1804b.

The user may apply the cover 1830 (e.g., glove) to their hand and loop an interior handle 1939 over the user's fingers, as shown in FIG. 20D. The user then pushes, or slides, the cover/glove under patient's side, as shown in FIG. 20C. For example, the user slides the cover 1830 between the procedure table and the patient. The hook material 1832 engages with the exterior of the endoscopy garment 1802 (e.g. at the anchor portion 1804b) and remains fixed in a position under the patient. The interior of the cover 1830 inverts and releases when the provider's hand moves away from the patient to expose an elastic band (e.g., 1936) with a section of hook material 1938. The user can pull the elastic band 1936 over the patient to generate localized compression. When the user has applied the desired amount of compression, the user secures the right side of the abdominal wrap (e.g., hook material 1938) to the anchor portion of the endoscopy garment 1802. FIG. 21A illustrates an example of the user using the handle to pull the elastic band 1936 from the interior of the cover 1830 after positioning the cover 1830 under the patient and securing the cover 1830 to anchor portion 1804b. FIG. 21B illustrates that the elastic band 1936 of the compression apparatus can be pull around the patient. FIG. 21C illustrates the end of the compression apparatus (e.g., hook material 1938) secured to the anchor portion 1804a of the endoscopy garment.

In some aspects, the cover 1830 may include a lower friction material, such as a satin, nylon, and/or polyester material. The elastic band 1936 may include a 4 inch knit elastic band, in some aspects. The compression apparatus may include polyester webbing, such as for the handle 1939. The compression apparatus may use a hook and loop material for the attachment to the anchor portions of the endoscopy garment. In some aspects, a neoprene material and/or a woven material may be used as a base for the endoscopy garment and/or the compression apparatus (or strap/wrap) that attaches to the endoscopy garment to apply targeted compression to the abdomen of the patient during an endoscopy procedure.

FIGS. 22A-24B illustrate an example in which the compression apparatus may be anchored to the procedure table at one end and to the endoscopy garment at the other end. FIG. 22A illustrates an example of a bar that can be mounted to the procedure table, e.g., by securing the mount portion 2204 to the underside of the table. Supports 2206 may extend between the mount portion 2204 and the bar to which the compression apparatus can attach. In some aspects, the bar may include one or more extensions 2208 that extend away from the support(s) 2206. The extensions 2208 may form a portion around which a loop 2232 formed at the end of the compression apparatus 2230 can be placed. The bar may include a flared portion 2202 that prevents the loop 2232 from sliding off the bar. In some aspects, multiple compression apparatuses may be attached to the bar, e.g., one on each side extension 2208. FIG. 22B illustrates a side view of the bar, and FIG. 22C illustrates a view of the bar having the compression apparatus looped around one extension.

In some aspects, the bar may include a steel handlebar mounted to the underside of procedure table. The compression apparatus 2230 may include an elastic compression band with a looped end that slides over the steel bar. In some aspects, the elastic band may be run, or stretched, from the bar over the table surface prior to patient arrival. FIG. 23 illustrates a view of a patient that is wearing a compression apparatus 2302 having at least one anchor portion 2304 and one or more reinforcement portions 2306. As shown in both views 2300 and 2350 in FIG. 23 and in FIG. 24A, the compression apparatus 2302 may be looped around the bar, and the patient may be positioned on top of the band of the compression apparatus (e.g., with the band between the patient and the procedure table 2310. If compression is required during the procedure, the compression apparatus (e.g., including an elastic band) can be looped over the patient and secured it to the anchor portion of the endoscopy garment worn by the patient. The steel bar and the attachment (e.g., with a hook and loop fastener) helps to brace the compression apparatus to create and hold localized compression. FIG. 24B illustrates that the compression apparatus may include a handle 2439, and a hook type material 2438, e.g., similar to the example described in connection with FIGS. 19C and 21A. In some aspects, the handlebar and mounting plate may include steel. In some aspects, the abdominal wrap may include one or more of a knit elastic band (e.g., a 4 inch knit elastic band), polyester webbing (e.g., for the handle 2439), a hook and/or loop material (such as Velcro®) to fasten or attach the compression apparatus to the anchor portion of the endoscopy garment. In some aspects, the compression apparatus may include a neoprene material.

FIG. 25 illustrates a flowchart showing example aspects of a method of applying compression in connection with an endoscopy procedure. As illustrated at 2502, the user positions a patient on a procedure table, the patient wearing a sleeve surrounding at least a portion of the abdomen of the patient, the sleeve comprising a front portion comprising a first material, a rear portion comprising a second material having a different elasticity than the front portion, and an anchor region comprising a loop material. The sleeve may include any of the aspects described in connection with any of FIGS. 2A-24B, for example.

At 2504, the user applies compression to an abdomen of the patient by stretching an elastic compression apparatus around at least a portion of the abdomen and securing a portion of the compression apparatus comprising a hook material to the anchor region of the sleeve. The compression device may include any of the aspects described in connection with any of FIGS. 2A-24B.

In some aspects, application of the compression may include placing an invertable cover of the compression apparatus over a hand of the user; sliding the invertable cover between the patient and the procedure table to cause a first section of the hook material to engage with a first anchor region of the sleeve; pulling an elastic band portion from an interior of the invertable cover to cause the invertable cover to at least partially invert; and stretching the elastic band portion around the at least the portion of the abdomen of the patient, wherein the hook material is secured to a second anchor region of the sleeve. For example, the method may include any of the aspects described in connection with FIGS. 13-17D.

In some aspects, the method further includes positioning a compression board between the patient and the procedure table, and securing a first end of the compression apparatus to the compression board prior to stretching the elastic band around the at least the portion of the abdomen of the patient. For example, the method may include any of the aspects described in connection with FIGS. 18A-21C.

In some aspects, the method may further include positioning a loop formed at one end of the compression apparatus around a fixed handle at the procedure table prior to stretching the elastic band around the at least the portion of the abdomen of the patient. For example, the method may include any of the aspects described in connection with FIGS. 22A-24B.

Example aspects of the present invention have now been described in accordance with the above advantages. It will be appreciated that these examples are merely illustrative of aspects of the present invention. Many variations and modifications will be apparent to those skilled in the art.

It is understood that the specific order or hierarchy of steps in the processes disclosed is an illustration of exemplary approaches. Based upon design preferences, it is understood that the specific order or hierarchy of steps in the processes may be rearranged. Further, some steps may be combined or omitted. The accompanying method claims present elements of the various steps in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects.” Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

The following aspects are illustrative only and may be combined with other aspects or teachings described herein, without limitation.

Aspect 1 is a an apparatus for applying pressure to an abdomen of a patient, comprising: a sleeve configured to surround at least a portion of the abdomen of the patient, the sleeve comprising: a front portion comprising a first material, arear portion comprising a second material having a different elasticity than the front portion, and one or more anchor regions on at least the front portion or the rear portion wherein a first anchor region of the one or more anchor regions along a left region of the sleeve is configured to removably couple with a first end of one or more compression apparatuses, wherein at least a second anchor region of the one or more anchor regions along the rear portion is configured to removably couple with a second end of each of the one or more compression apparatuses when placed over the abdomen of the patient between the first anchor region and at least the second anchor region.

In aspect 2, the apparatus of aspect 1, further comprises the one or more compression apparatuses, each of the one or more compression apparatuses comprising the first end, a body, and the second end, wherein the first end is configured to removably couple to the first anchor region, and when the second end is coupled to at least the second anchor region of the sleeve, the one or more compression apparatuses extended over the abdomen of the patient to apply a pressure upon at least a portion of the abdomen of the patient.

In aspect 3, the apparatus of aspect 1 or aspect 2 further includes that the first end or the second end of the one or more compression apparatuses comprise a fastener, wherein the first end couples to the first anchor region or the second end couples to at least the second anchor region, wherein the body of the one or more compression bands applies the pressure upon the portion of the abdomen of the patient when the first end is received at the first anchor region and the second end is received by at least the second anchor region.

In aspect 4, the apparatus of any of aspects 1-3 further includes that at least the first end or the second end of the one or more compression apparatuses comprise a handle opposite the fastener, wherein the handle configured to assist with placement of the first end with the first anchor region or the second end with the at least the second anchor region.

In aspect 5, the apparatus of any of aspects 1-4 further includes that the body of the one or more compression apparatuses is configured to apply the pressure upon a desired portion of the abdomen of the patient based on a positioning at which the second end is coupled onto at least the second anchor region.

In aspect 6, the apparatus of any of aspects 1-5 further includes that the one or more anchor regions are configured to adjust a localized pressure onto the desired portion of the abdomen of the patient based on an adjustment of the positioning of the second end of the one or more compression apparatuses onto at least the second anchor region of the sleeve.

In aspect 7, the apparatus of any of aspects 1-6 further includes that the rear portion of the sleeve covers a back region of the patient, wherein the rear portion of the sleeve comprises a second material comprised of a less elastic material than the front portion to maintain or align the rear portion of the sleeve onto the back region of the patient.

In aspect 8, the apparatus of any of aspects 1-7 further includes that the second material of the rear portion of the sleeve is configured to provide a support structure to counter pressure applied by the one or more compression bands to maintain or align the rear portion of the sleeve with the back region of the patient.

In aspect 9, the apparatus of any of aspects 1-8 further includes that the rear portion of the sleeve comprises at least the second anchor region, wherein the second end of the one or more compression apparatuses are received by at least the second anchor region of the rear portion of the sleeve.

In aspect 10, the apparatus of any of aspects 1-9 further includes that the front portion of the sleeve comprises the one or more anchor regions, wherein the second end of the one or more compression apparatuses are received by the one or more anchor regions of the front portion of the sleeve.

In aspect 11, the apparatus of any of aspects 1-10 further includes that the sleeve comprises a unitary sleeve, wherein the unitary sleeve is an endoscopy garment that is fastened around the patient and provides access to a rectum of the patient.

In aspect 12, the apparatus of any of aspects 1-11 further includes that the endoscopy garment extends around a groin region of the patient, wherein the endoscopy garment extended around the groin region provides support for the unitary sleeve to receive the one or more compression apparatuses.

In aspect 13, the apparatus of any of aspects 1-12 further includes that the endoscopy garment further comprises one or more shoulder straps that wrap around a shoulder region of the patient, wherein the endoscopy garment wrapped around the shoulder region of the patient provides support for the one or more compression apparatuses.

In aspect 14, the apparatus of any of aspects 1-13 further includes that the first anchor region extends along the left region of the sleeve provides a surface to receive the first end of the one or more compression apparatuses.

In aspect 15, the apparatus of any of aspects 1-14 further includes that the surface of the first anchor region extends along the left region of the sleeve such that the first end of the one or more compression apparatuses is removably coupled to the surface of the first anchor region.

In aspect 16, the apparatus of any of aspects 1-15 further includes that the first anchor region on the left region of the sleeve is arranged proximate a left hip region of the patient.

In aspect 17, the apparatus of any of aspects 1-16 further includes that the surface of the first anchor region comprises a fastener that corresponds with the first end of the one or more compression bands to removably couple the one or more compression apparatuses to the first anchor region.

In aspect 18, the apparatus of any of aspects 1-7 further includes at least one attachment extension proximate the front portion or the rear portion, wherein the at least one attachment extension extends along the left region of the sleeve and is configured to removably couple with a first end of the one or more compression apparatuses.

In aspect 19, the apparatus of any of aspects 1-18 further includes that the at least one attachment extension comprises a plurality of attachment extensions that extend along at least the portion of the sleeve.

In aspect 20, the apparatus of any of aspects 1-19 further includes that the plurality of attachment extensions forms a v-shaped groove, wherein the first end of the one or more compression apparatuses is received by the v-shaped groove to removably couple the first end of the one or more compression apparatuses to the plurality of attachment extensions.

In aspect 21, the apparatus of any of aspects 1-20 further includes a compression apparatus that is removably attachable to the one or more anchor regions of the sleeve, wherein the compression apparatus includes: an invertable cover having an opening sized to receive a hand of a user; an elastic band coupled to an interior of the cover opposite the opening; a first hook material portion; a second hook material portion; and a handle.

In aspect 22, the apparatus of any of aspects 1-21 further includes a compression board having one or more openings to receive a hook provided at the end of the one or more compression apparatuses.

In aspect 23, the apparatus of any of aspects 1-22 further includes that a compression apparatus has a loop formed at one end to receive a fixed handle at a procedure table and a hook material at a second end to removably couple to an anchor portion of the one or more anchor portions.

Aspect 24 is a method of method of applying compression during an endoscopy procedure, comprising: positioning a patient on a procedure table, the patient wearing a sleeve surrounding at least a portion of the abdomen of the patient, the sleeve comprising a front portion comprising a first material, a rear portion comprising a second material having a different elasticity than the front portion, and an anchor region comprising a loop material; and applying compression to an abdomen of the patient by stretching an elastic compression apparatus around at least a portion of the abdomen and securing a portion of the compression apparatus comprising a hook material to the anchor region of the sleeve.

In aspect 25, the method of aspect 24 further includes that applying the compression includes: placing an invertable cover of the compression apparatus over a hand of the user; sliding the invertable cover between the patient and the procedure table to cause a first section of the hook material to engage with a first anchor region of the sleeve; pulling an elastic band portion from an interior of the invertable cover to cause the invertable cover to at least partially invert; and stretching the elastic band portion around the at least the portion of the abdomen of the patient, wherein the hook material is secured to a second anchor region of the sleeve.

In aspect 26, the method of aspect 24 or 25 further includes positioning a compression board between the patient and the procedure table; and securing a first end of the compression apparatus to the compression board prior to stretching the elastic band around the at least the portion of the abdomen of the patient.

In aspect 27, the method of any of aspects 24-26 further includes positioning a loop formed at one end of the compression apparatus around a fixed handle at the procedure table prior to stretching the elastic band around the at least the portion of the abdomen of the patient.

1. An apparatus for applying pressure to an abdomen of a patient, comprising:
a sleeve configured to surround at least a portion of the abdomen of the patient, the sleeve comprising:
a front portion comprising a first material,
a rear portion comprising a second material having a different elasticity than the front portion, and
one or more anchor regions on at least the front portion or the rear portion wherein a first anchor region of the one or more anchor regions along a left region of the sleeve is configured to removably couple with a first end of one or more compression apparatuses, wherein at least a second anchor region of the one or more anchor regions along the rear portion is configured to removably couple with a second end of each of the one or more compression apparatuses when placed over the abdomen of the patient between the first anchor region and at least the second anchor region.

2. The apparatus of claim 1, further comprising the one or more compression apparatuses, each of the one or more compression apparatuses comprising the first end, a body, and the second end, wherein the first end is configured to removably couple to the first anchor region, and when the second end is coupled to at least the second anchor region of the sleeve, the one or more compression apparatuses extended over the abdomen of the patient to apply a pressure upon at least a portion of the abdomen of the patient.

3. The apparatus of claim 2, wherein the first end or the second end of the one or more compression apparatuses comprise a fastener, wherein the first end couples to the first anchor region or the second end couples to at least the second anchor region, wherein the body of the one or more compression apparatuses applies the pressure upon the portion of the abdomen of the patient when the first end is received at the first anchor region and the second end is received by at least the second anchor region.

4. The apparatus of claim 3, wherein at least the first end or the second end of the one or more compression apparatuses comprise a handle opposite the fastener, wherein the handle configured to assist with placement of the first end with the first anchor region or the second end with the at least the second anchor region.

5. The apparatus of claim 2, wherein the body of the one or more compression apparatuses is configured to apply the pressure upon a desired portion of the abdomen of the patient based on a positioning at which the second end is coupled onto at least the second anchor region.

6. The apparatus of claim 5, wherein the one or more anchor regions are configured to adjust a localized pressure onto the desired portion of the abdomen of the patient based on an adjustment of the positioning of the second end of the one or more compression apparatuses onto at least the second anchor region of the sleeve.

7. The apparatus of claim 1, wherein the rear portion of the sleeve covers a back region of the patient, wherein the rear portion of the sleeve comprises a second material comprised of a less elastic material than the front portion to maintain or align the rear portion of the sleeve onto the back region of the patient.

8. The apparatus of claim 7, wherein the second material of the rear portion of the sleeve is configured to provide a support structure to counter pressure applied by the one or more compression appartuses to maintain or align the rear portion of the sleeve with the back region of the patient.

9. The apparatus of claim 7, wherein the rear portion of the sleeve comprises at least the second anchor region, wherein the second end of the one or more compression apparatuses are received by at least the second anchor region of the rear portion of the sleeve.

10. The apparatus of claim 1, wherein the front portion of the sleeve comprises the one or more anchor regions, wherein the second end of the one or more compression apparatuses are received by the one or more anchor regions of the front portion of the sleeve.

11. The apparatus of claim 1, wherein the sleeve comprises a unitary sleeve, wherein the unitary sleeve is an endoscopy garment that is fastened around the patient and provides access to a rectum of the patient.

12. The apparatus of claim 11, wherein the endoscopy garment extends around a groin region of the patient, wherein the endoscopy garment extended around the groin region provides support for the unitary sleeve to receive the one or more compression apparatuses.

13. The apparatus of claim 12, wherein the endoscopy garment further comprises one or more shoulder straps that wrap around a shoulder region of the patient, wherein the endoscopy garment wrapped around the shoulder region of the patient provides support for the one or more compression apparatuses.

14. The apparatus of claim 1, wherein the first anchor region extends along the left region of the sleeve provides a surface to receive the first end of the one or more compression apparatuses.

15. The apparatus of claim 14, wherein the surface of the first anchor region extends along the left region of the sleeve such that the first end of the one or more compression apparatuses is removably coupled to the surface of the first anchor region.

16. The apparatus of claim 14, wherein the first anchor region on the left region of the sleeve is arranged proximate a left hip region of the patient.

17. The apparatus of claim 14, wherein the surface of the first anchor region comprises a fastener that corresponds with the first end of the one or more compression apparatuses to removably couple the one or more compression apparatuses to the first anchor region.

18. The apparatus of claim 1, further comprising:
at least one attachment extension proximate the front portion or the rear portion, wherein the at least one attachment extension extends along the left region of the sleeve is configured to removably couple with a first end of the one or more compression apparatuses.

19. The apparatus of claim 18, wherein the at least one attachment extension comprises a plurality of attachment extensions that extend along at least the portion of the sleeve.

20. The apparatus of claim 19, wherein the plurality of attachment extensions forms a v-shaped groove, wherein the first end of the one or more compression apparatuses is received by the v-shaped groove to removably couple the first end of the one or more compression apparatuses to the plurality of attachment extensions.

21. The apparatus of claim 1, further including a compression apparatus that is removably attachable to the one or more anchor regions of the sleeve, wherein the compression apparatus includes:
an invertable cover having an opening sized to receive a hand of a user;
an elastic band coupled to an interior of the invertable cover opposite the opening;
a first hook material portion;
a second hook material portion; and
a handle.

22. The apparatus of claim 1, further including a compression board having one or more openings to receive a hook provided at the end of the one or more compression apparatuses.

23. The apparatus of claim 1, further including a compression apparatus having a loop formed at one end to receive a fixed handle at a procedure table and a hook material at a second end to removably couple to an anchor portion of the sleeve.

24. A method of applying compression during an endoscopy procedure, comprising:
positioning a patient on a procedure table, the patient wearing a sleeve surrounding at least a portion of an abdomen of the patient, the sleeve comprising a front portion comprising a first material, a rear portion comprising a second material having a different elasticity than the front portion, and an anchor region comprising a loop material; and
applying the compression to an abdomen of the patient by stretching an elastic compression apparatus around at least a portion of the abdomen and securing a portion of the compression apparatus comprising a hook material to the anchor region of the sleeve.

25. The method of claim 24, wherein applying the compression includes:
placing an invertable cover of the compression apparatus over a hand of a user;
sliding the invertable cover between the patient and the procedure table to cause a first section of the hook material to engage with a first anchor region of the sleeve;
pulling an elastic band portion from an interior of the invertable cover to cause the invertable cover to at least partially invert; and
stretching the elastic band portion around the at least the portion of the abdomen of the patient, wherein the hook material is secured to a second anchor region of the sleeve.

26. The method of claim 24, further comprising:
positioning a compression board between the patient and the procedure table; and
securing a first end of the compression apparatus to the compression board prior to stretching the elastic band portion around the at least the portion of the abdomen of the patient.

27. The method of claim 24, further comprising:
positioning a loop formed at one end of the compression apparatus around a fixed handle at the procedure table prior to stretching the elastic band portion around the at least the portion of the abdomen of the patient.
